(19) United States
(12) Patent Application Publication
(10) Pub. No.: US 2025/0341829 A1
(43) Pub. Date: Nov. 6, 2025

(54) OIL PROGNOSTIC TOOL AND METHOD

(71) Applicant: Spectro Scientific, Inc., Chelmsford, MA (US)

(72) Inventor: Daniel Kienle, Boston, MA (US)

(21) Appl. No.: 19/192,682

(22) Filed: Apr. 29, 2025

Related U.S. Application Data
(60) Provisional application No. 63/641,686, filed on May 2, 2024.

(51) Int. Cl. G05B 23/02; G01N 33/28

(52) U.S. Cl. CPC G05B 23/0283; G01N 33/2835; G01N 33/2888; G05B 23/0227

(57) ABSTRACT

An on-site oil analysis method and system employs a rule-based diagnostic system to generate historical oil condition indicators for an asset used to model future oil condition indicators including probabilistic trajectories of continued oil degradation. Based on a user risk tolerance level, a modeled future oil condition indicator which will result in oil failure is determined. The oil condition indicator which will result in oil failure first and when is detected and provided as an output.

### RELATED APPLICATIONS

This application claims benefit of and priority to U.S. Provisional Application Ser. No. 63/641,686 filed May 2, 2024, under 35 U.S.C. §§ 119, 120, 363, 365, and 37 C.F.R. § 1.55 and § 1.78, which is incorporated herein by this reference.

### FIELD OF THE INVENTION

This subject invention relates to oil analysis.

### BACKGROUND OF THE INVENTION

In-service asset oil analysis is an important component of condition-based maintenance (CBM). As a part of CBM, the measured properties of oils sampled from operating machines are typically tested against a set of threshold values to determine if the oil is suitable for continued operation and to diagnose any problems with the machine operation such as leaks, excessive contamination, or wear. Because decisions about oil changes and maintenance are based on the current condition of the oil, CBM relies on sampling at regular intervals which can have unnecessary costs associated with oversampling and the possibility of operating on severely degraded oil between samples which can result in damage to the machine.

One solution to this shortcoming is to estimate the remaining useful life (RUL) of the oil over the course of its life as an indication of when the oil will need to be changed. This can be accomplished by tracking trends in oil properties that are used to indicate oil condition (oil condition indicators, OCIs), and using the trend to estimate when the OCI will cross a failure threshold value. This information can be used to avoid the costs associated with unplanned/corrective maintenance.

Most previous patents for determining the RUL of lubricants have been designed for continuous or intermittent in-situ monitoring of the OCIs in real time (see, for example, U.S. Pat. No. 11,175,274; WO 2023/278434; U.S. Pat. No. 6,741,938; 2019/0195097; U.S. Pat. No. 7,355,415; and 6,253,601 all incorporated herein by this reference).

Limitations include the fact that the set of analytical methods that can be performed in-situ are restricted to available in-situ sensor technology and that the methods are only useable in machines equipped with the required sensors. Another shortcoming of the prior art is that they produce point estimates of RUL based on data collected since the most recent oil change and thus do not account for the random nature of oil degradation resulting from variation in operating conditions (e.g., different operators, variation in weather or demand, etc.) that can cause significant uncertainty in the exact value for the RUL. For example, WO 2023/278434; U.S. Pat. No. 7,355,415; 5,750,887; US 2010/0250156; U.S. Pat. No. 7,581,434; US 2017/0044942; and U.S. Pat. No. 11,175,274 (all incorporated herein by this reference) describe systems that estimate RUL based on fitting a model of oil degradation to in-situ OCI measurements and extrapolating the best fit to a failure threshold, while U.S. Pat. No. 6,741,938 and 6,253,601 describe systems that predict RUL using static models based on the operating time since an oil change or oil life event. The absence of an accurate assessment of the RUL probability distribution in prior art precludes useful analyses such as maintenance optimization and estimation of the probability of oil failure before the end of a mission. Furthermore, the risk of oil failure corresponding to a point estimate of RUL is 50%. If, in reality, the RUL has a large degree of uncertainty due to variable operating conditions, the machine could be operating at an elevated risk of oil failure, approaching 50%, well before reaching the RUL prescribed based on a point estimate.

More sophisticated methods for RUL estimation of oil are described in academic literature. While these applications do estimate the RUL probability distribution, they do so by using arbitrary and fixed descriptions of random variation in degradation rate over time (See J. Echauz, A. Gardner, R. R. Curtin, N. Vasiloglou, and G. Vachtsevanos, “PFsuper: Simulation-Based Prognostics to Monitor and Predict Sparse Time Series”, Annual Conference of the PHM Society, vol. 9, no. 1, 2017, doi: 10.36001/phmconf.2017.v9i1.2481) (i.e., process noise) and ignoring epistemic uncertainty by using point estimates (i.e., maximum likelihood estimation) for the model parameters (See J. Echauz, A. Gardner, R. R. Curtin, N. Vasiloglou, and G. Vachtsevanos, “PFsuper: Simulation-Based Prognostics to Monitor and Predict Sparse Time Series”, Annual Conference of the PHM Society, vol. 9, no. 1, 2017, doi: 10.36001/phmconf.2017.v9i1.2481; J. Zhu, J. Yoon, D. He, Y. Qu, E. Bechhoefer, “Lubrication Oil Condition Monitoring and Remaining Useful Life Prediction with Particle Filtering”, International Journal of Prognostics and Health Management, vol. 4, no. 3, 2013, https://doi.org/10.36001/ijphm.2013.v413.2151; Y. Pan, Z. Han, T. Wu and Y. Lei, “Remaining Useful Life Prediction of Lubricating Oil With Small Samples”, IEEE Transactions on Industrial Electronics, vol. 70, no. 7, 2023, doi: 10.1109/TIE.2022.3201289). While this approximation can produce reasonable predictions for linear degradation models with small process noise, short projections, and large data sets, it can have a severe impact on the RUL distribution for nonlinear models with significant process noise, long projections, and/or small data sets. In the case of oil analysis, process noise is often significant due to varying operating conditions (e.g., weather, load, human operator), especially for measurement of viscosity and wear debris, and sampling rates are low. Resulting inaccuracy in the RUL distribution can lead to a nonoptimal maintenance strategy and increased risk of operation with degraded oil.

Finally, both the cited patents and examples from academic literature depend only on data collected since the most recent oil change from a single asset instead of using the full history of a fleet of assets to train and update projections. This approach severely limits the degradation characteristics that can be described by the model, given that only a few samples are collected between oil changes, typically. For example, age dependence, seasonality, general non-linearity, and probabilistic anomalies could not be accurately characterized by such a small data set collected over a recent history.

### BRIEF SUMMARY OF THE INVENTION

The proposed tool (henceforth referred to as “Prognostics”) overcomes the described shortcomings of previous systems and methods and provides additional functionality. First, Prognostics is designed for use with sparsely sampled, non-uniform data from on-site oil analytical instruments. The practice of on-site oil analysis using portable instruments is more widely accessible than in-situ measurement and includes a richer array of analytical techniques, including chemical analysis via infrared spectroscopy, viscometry, densitometry, x-ray fluorescence spectroscopy for elemental analysis of large wear particles, optical emission spectroscopies (OES) such as rotating disk electrode (RDE) or spark OES for elemental analysis of small or soluble wear components, and particle analysis via laser net fines. See U.S. Pat. No. 11,796,489 incorporated herein by this reference.

One drawback of on-site oil analysis is that it relies on manual data entry, which is prone to error, rather than streaming data directly from an on-board computer. Prognostics manages this with built-in filtering steps and data quality checks to detect anomalous measurements and inaccuracies in the manual data entry. A second distinction between Prognostics and prior art is the rigorous analytical methods used. Preferably, rather than producing a point estimate of RUL using OCI data only taken from a single asset since the previous oil change, Prognostics incorporates all available historical OCI data from a fleet of assets to train a probabilistic model of oil degradation. This approach allows long-term trends in oil degradation that depend on the time of year or asset age to be detected and incorporated in the degradation model. Likewise, the process noise distribution can be determined (parameterized) from the data as a part of training or updating the degradation model. Once trained, the model is used to estimate the probability of failure over time with continued operation as well as the probability that each new measurement represents abnormal operation. This information is used to provide risk-optimized estimates of when the oil next needs to be measured, when an oil change will be required, and when the oil is expected to cross into the severely degraded state, as well as to detect and flag measurements that may indicate faulty operation or unexpected changes to operating conditions.

The additional information provided by Prognostics in some examples offers several key advantages over CBM programs that rely exclusively on rule-based diagnostics and existing RUL estimation methods that do not offer risk optimized scheduling. First, the ability to forecast a risk-optimized preventative maintenance schedule avoids costs associated with unplanned/corrective maintenance, in addition to labor, equipment, consumable, and downtime costs associated with unnecessary lubricant sampling and premature oil change. Second, this scheduling information is necessary for logistical optimization of plant or fleet maintenance operations. Finally, flagging anomalous wear, contamination, or chemical results relative to asset or fleet historical trends has the potential to provide a warning of detrimental changes in operating conditions or even incipient component failure before the oil crosses a diagnostic threshold.

An on-site oil analysis method and system employs a rule-based diagnostic system to generate historical oil condition indicators for an asset used to model future oil condition indicators including probabilistic trajectories of continued oil degradation. Based on a user risk tolerance level, a modeled future oil condition indicator which will result in oil failure is determined. The oil condition indicator which will result in oil failure first and when is detected and provided as an output.

In one embodiment, modeling future oil condition indicators include employing a stochastic degradation model. In one example, the oil condition indicators include a Si concentration, an Fe concentration, a Cu concentration, a Na concentration, a Pb concentration, an Al concentration, a Cr concentration, viscosity, particle count, water concentration, oxidation, TAN, and/or TBN.

The rule based diagnostic system may also generate historical oil change data. The system and method may further determine and output when a new measurement is required for an oil condition indicator and when an oil change is recommended.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is an exemplary flow diagram illustrating the new data processing steps of a new system including the relative role of a rule-based diagnostics algorithm the oil prognostics tool;

FIGS. 2A-2J are graphical demonstrations of the prognostic analysis of Si (FIGS. 2A-2B) concentration, Fe concentration (FIGS. 2C-2D) Cu concentration, (FIGS. 2E-2F) oxidation (FIGS. 2G-2H) and TBN (FIGS. 2I-2J) from the engine oil of a Komatsu HD785-7 dump truck. FIGS. 2A, 2C, 2E, 2G, and 2I show the measured properties versus miles operating of the asset (circles) over multiple oil change cycles (indicated by vertical dashed lines). The grayscale lines show possible trajectories of continued degradation, and the horizontal dashed line indicates the failure threshold. FIGS. 2B, 2D, 2F, 2H, and 2J show the probability of failure with continued operation (solid curve) and the risk-tolerance for oil failure (horizontal dotted line). The risk-optimized miles before a new measurement is required, the most likely oil change mileage, and the mileage where oil failure is expected are indicated by the dotted vertical black and gray lines and the vertical bold gray line, respectively;

FIG. 3 is an example of text results that are returned by Prognostics software tool when used to analyze the engine oil data from the Komatsu HD785-7 dump truck; and

FIG. 4 is an example of graphical timelines showing the optimal maintenance schedule for nine dump trucks, and is a representation of how the results may be displayed to the user graphically.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

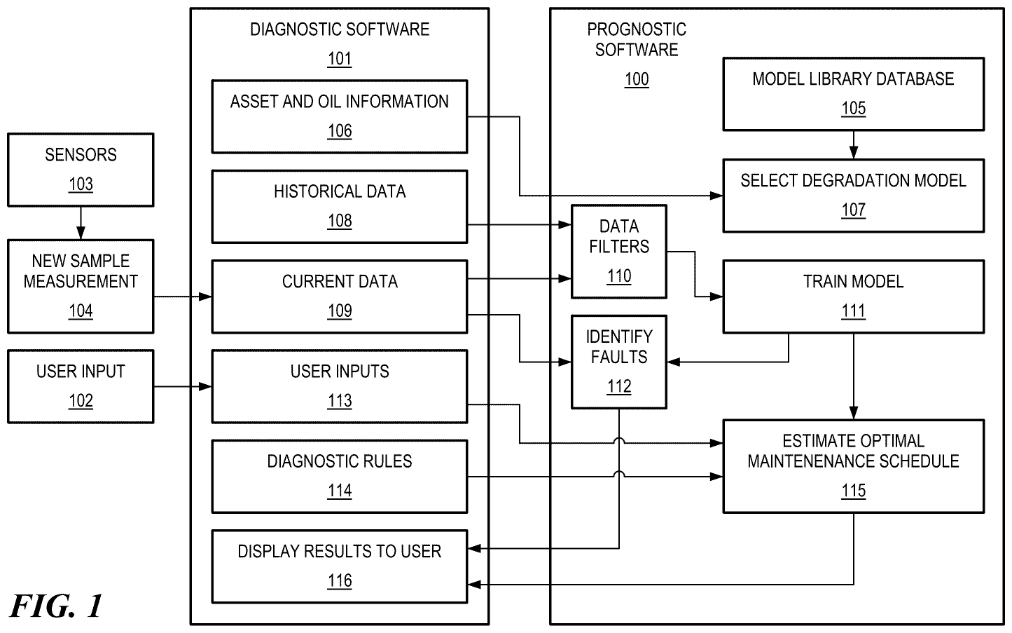

FIG. 1 is a flow diagram of an exemplary data processing method including the relative role of the Prognostics software 100 and the supporting rule-based diagnostic software program 101 (such as TruVu360®), which contains the data and parameters necessary for analysis including historical 108 and current 109 lubricant property data (e.g., TAN, TBN, Oxidation, contaminant concentrations, and elemental concentrations from wear particles or additives), the type of component and oil being measured 106, the threshold for severe degradation of each property 114 and any additional user inputs 113. Software 100 includes means (e.g., computer instructions) for modeling future oil condition indicators including probabilistic trajectories of continued oil degradation using said historical oil condition indicators, for determining, based on a user risk tolerance level, when a modeled future oil condition indicator will result in oil failure, for detecting the oil condition indicator which will result in oil failure first and when, and for outputting at least the determined oil condition indicator which will result in oil failure first and when. Prognostics may require prerequisite one-time user input of additional parameters 102 which are also stored in the diagnostic software and include risk tolerance for severe oil degradation, typical oil change interval, and typical sampling interval. Optional one-time user input parameters may also be entered to enable additional features such as fleet level prognostics, cost-based analysis, and estimates of cost savings achieved through the use of Prognostics.

Preferably Prognostics does not require additional user input with each oil sample because it can interface with diagnostic software such as TruVu360® to automatically run when each sample is analyzed. Specifically, Prognostics analysis may be triggered when a new sample is measured on oil analysis instrumentation 103 and uploaded to the diagnostic software, 104. The first step, 107, in the analysis is to select a degradation model from the model library 105 stored in the Prognostics software using information about the measured asset type and the oil sampled, 106, provided by the diagnostic software. Next, the diagnostic software provides the OCIs and lifetimes of the newly measured sample, 109, and the historical samples from the asset and similar assets, 108, to Prognostics. Prognostics then filters (110) the data set for human or experimental anomalies. The data is used to train or update a probabilistic model 111 of the degradation and state of the oil. The trained model is first used to detect any probabilistic faults 112 of the current measurement that could indicate a problem with the asset. It is then used to estimate, step 115, the survival time distribution to define the optimal maintenance schedule given the user inputs 113 (e.g., risk tolerance) and the diagnostic rules 114 including failure threshold values for each OCI, which are provided by the diagnostic software. The results of the analysis including warning messages about detected faults, and the prescribed maintenance schedule are provided by Prognostics to the diagnostic software for displaying to the user, step 116.

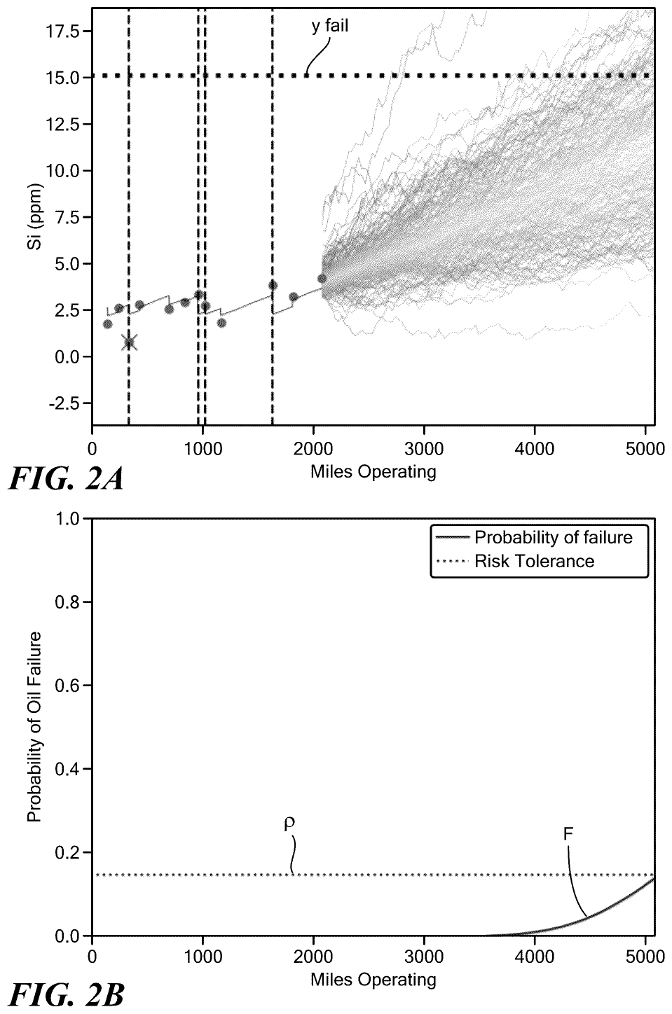

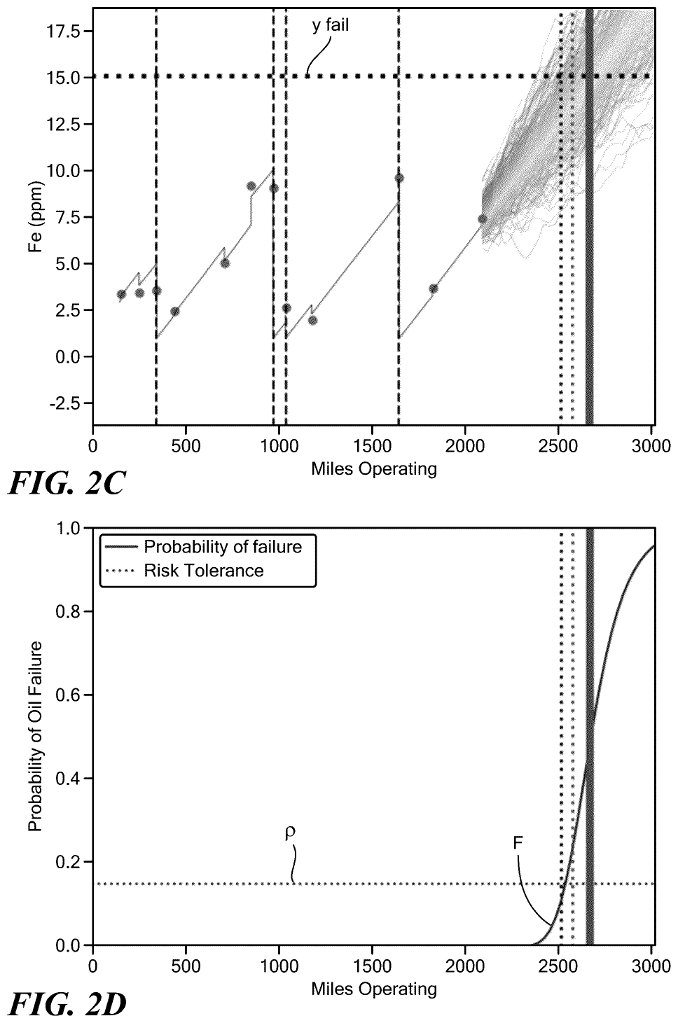

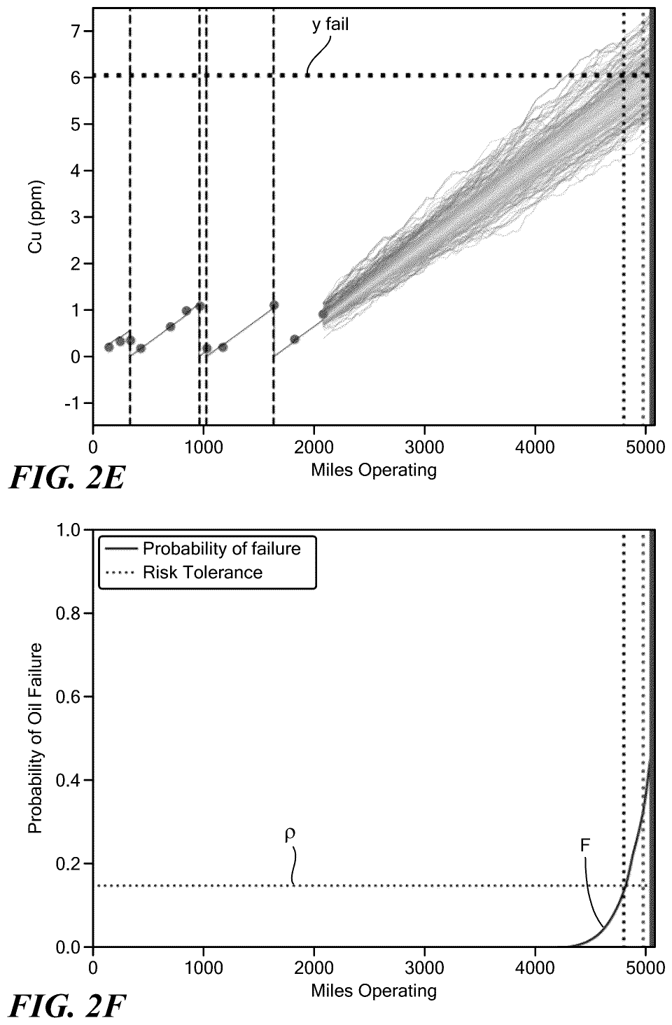

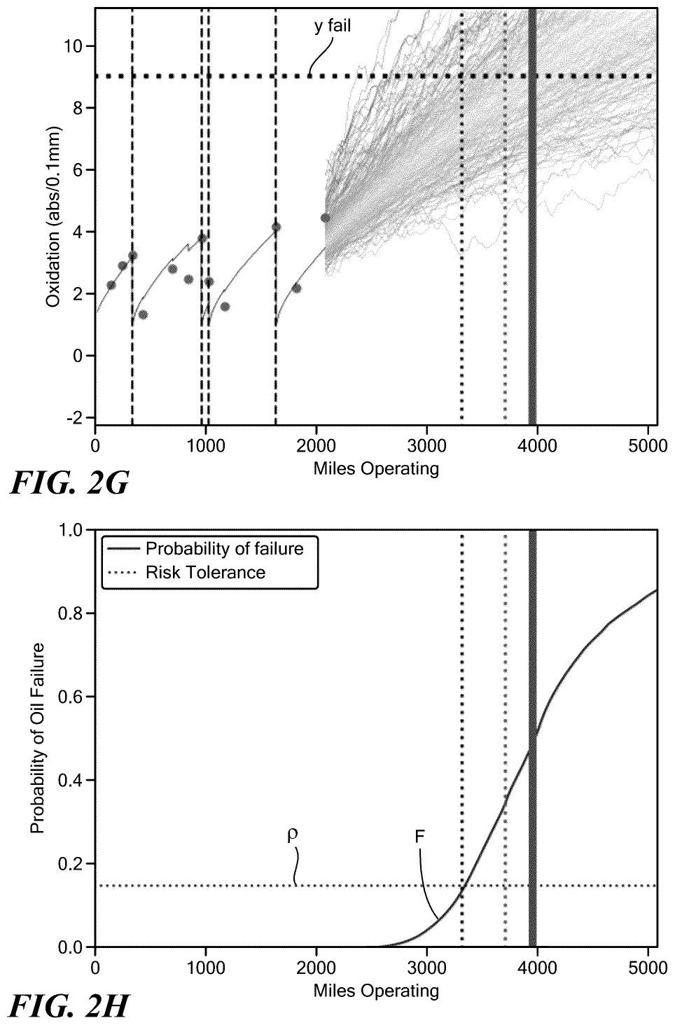

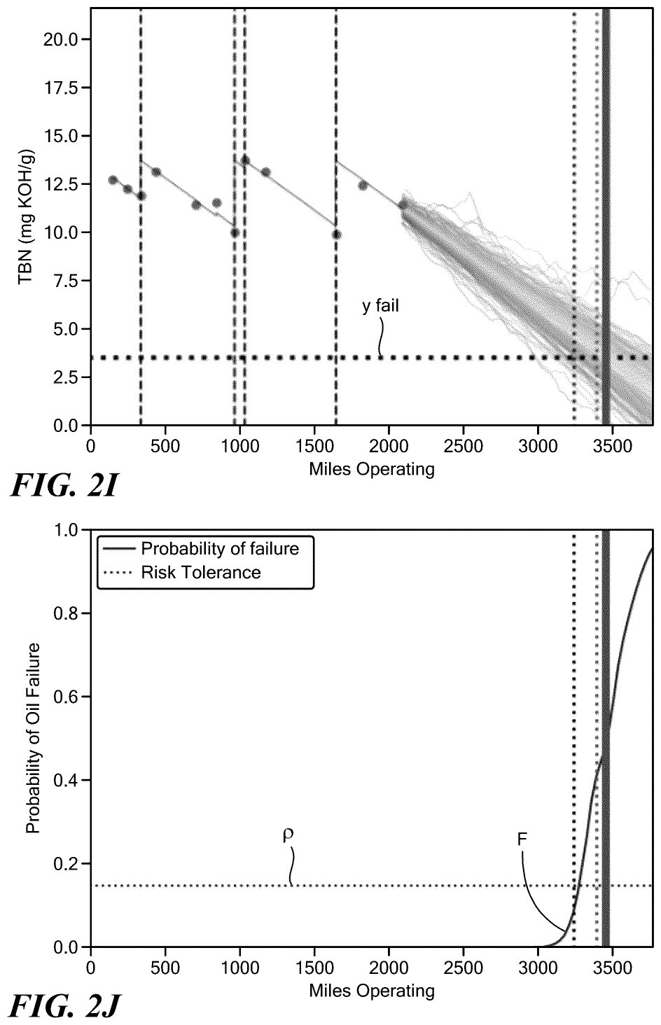

As a demonstration, Prognostics software was used to estimate the mileages corresponding to expected failure, next sample draw, and the next oil change for the engine oil of a Komatsu HD785-7 dump truck. It provided the estimates by analyzing historical values for Si, Fe, and Cu concentrations (FIGS. 2A-F, respectively) from RDE OES, and oxidation and TBN (FIGS. 2G-J, respectively) from IR spectroscopy. Graphical and text results are shown as in FIGS. 2-4, and a stepwise summary of the procedure Prognostics performs after each measurement is provided below.

The system receives historical data y(t_{1:k}) (datapoints of FIGS. 2A, 2C, 2E, 2G, and 2I), thresholds used to define severe degradation predefined by diagnostic software y_{fail} (horizontal dashed lines of FIGS. 2A, 2C, 2E, 2G, and 2I), reference values, component type, oil type, and user-defined risk tolerance ρ (horizontal dashed lines of FIGS. 2B, 2D, 2F, 2H, and 2G) from a rule-based diagnostic software, such as TruVu360® (spectrosci.com/product/truvu-360).

The system selects a stochastic degradation model, step 107, FIG. 1, with characteristics expected of the component type (e.g. heavy-duty diesel engine) and oil type. The model takes the form of the stochastic, differential equation,

$$y ( t ) = h ( x ( t ) ) + μ , and \qquad ( 1 )$$

$$\frac{d x ( t )}{d t} = g ( x ( t ) , θ , t ) + υ . \qquad ( 2 )$$

Here, y(t) and x(t) are the OCI and latent state (e.g. the true property value in the absence of measurement noise or an unmeasured property being calculated from measured properties) at time or distance, t, respectively. The function h relates x to y, and g is the deterministic component of the dynamic model. The variables μ, and v are the measurement noise, and process noise, respectively, and are sampled from probability distributions of arbitrary type. Finally, the stationary parameters are held in θ. Many OCIs have linear degradation trends (FIGS. 2A, 2C, 2E, and FIG. 2H), however some have higher order degradation trends as shown in FIG. 2G, while others may have more complex dynamics such as oscillations, transitioning between multiple discrete dynamic models (e.g. wear modes), or interdependence between multiple oil properties.

The system then trains or updates the model to estimate the joint posterior likelihood, P(x(tₖ), θ|y(t_{1:k})) at the time (or distance) of the current measurement, tₖ, 111, FIG. 1.

The system uses the trained model to estimate the optimized maintenance schedule, step 115. To do this, the system samples a comprehensive set of x(tₖ) and θ values from P(x(tₖ),θ|y(t_{1:k})) using importance sampling and uses each sample with equations 1-2 to simulate possible trajectories of continued degradation of y (grayscale lines in FIG. 2) and uses the time or distance to failure of each trajectory, t_{fail} (i.e., when the trajectory crosses the failure threshold), to define the cumulative probability of failure with continued operation for each model as

$$F ( {t}_{fail} \leq t ) = \frac{{N}_{failed} ( t )}{N} ,$$

where N_{failed}(t) is the number of trajectories that have failed prior to time or distance, t, and N is the total number of trajectories, FIGS. 2B, 2D, 2F, 2H, and 2J. This is equivalent to the cumulative RUL distribution and accounts for uncertainty in the model.

The system then defines the most likely cumulative probability of failure with continued operation, \hat{F}(t_{fail}≤t) for each model using only the set of x(tₖ) and θ that maximizes P(x(tₖ),θ|y(t_{1:k})) (i.e. the best fit to the model). This represents the most likely path to failure and an estimate of when the oil will fail but does not account for uncertainty in the model and the corresponding increase in risk.

At step 112, FIG. 1, the system determines if the current measurement is anomalous (indicated by open circles and x's in FIG. 2). A measurement is defined as anomalous if the probability of the measured value, P(y(tₖ)|y(t_{1:k-1}))=∫∫[P_{μ}(y(tₖ)/h(x(tₖ)*P(x(tₖ),θ|y(t_{1:k-1}))]dθdx(tₖ), is less than a fixed threshold.

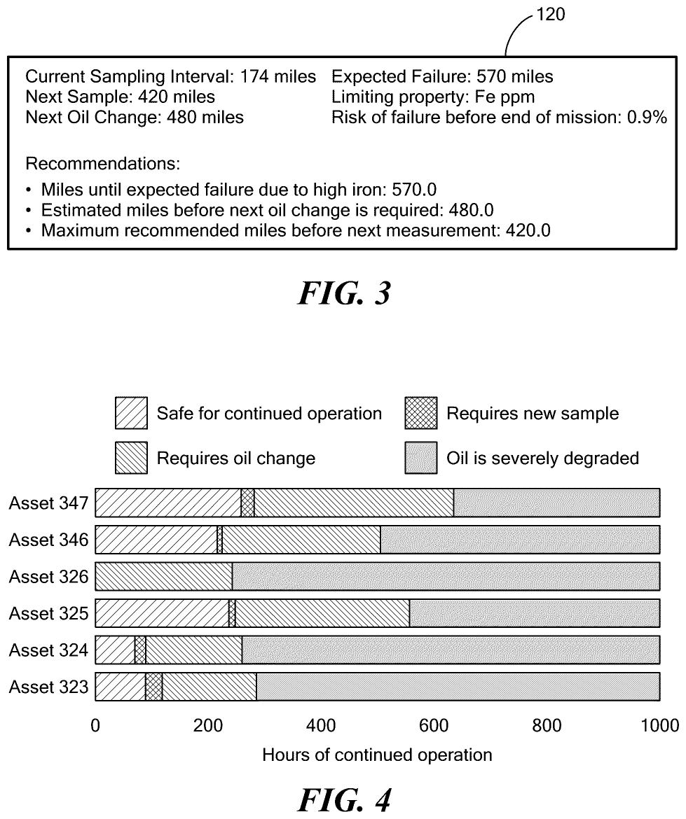

The system may define the following results shown at 120, FIG. 3 for each model: The current sampling interval: Average time or distance between samples in historical data; Next oil change: The time or distance until the risk tolerance will be exceeded by the most likely probability of failure, ignoring uncertainty in the model parameters (vertical gray dotted line in FIG. 2), \hat{t}_{ρ}−tₖ, where \hat{t}_{ρ}=argₜ{\hat{F}(t_{fail}≤t)=ρ}; Next

Sample: The time or distance until the risk tolerance is exceeded by the probability of failure while accounting for uncertainty in the model parameters (vertical black dotted line in FIG. 2), t₉₂ −tₖ, where t_{ρ}=argₜ{F(t_{fail}≤t)=ρ}; Expected failure: The time or distance until the oil has a 50% chance of failure (bold vertical gray lines in FIG. 2), t_{EF}−tₖ, where t_{EF}=argₜ{F(t_{fail}≤t)=0.5}; and Risk of failure before end of mission is the probability that the oil will fail before the end of the mission or next scheduled sampling of the oil, F(t_{fail}≤t_{EOM}) where t_{EOM} is the operating time or distance at which the mission is expected to be complete and is defined by the user.

The system reports the minimum value for each of the listed results across the analyzed properties, step 116, FIG. 1 (and messaging and parameter values shown in FIG. 3). In this case the Fe concentration was estimated to be the first to fail, so the reported values correspond to estimates from modeling Fe concentration shown in FIGS. 2C-2D.

The system reports any properties or combinations of properties that are exhibiting anomalous degradation characteristics. In this example, none of the current measurements were found to be anomalous, so no warnings are issued.

In addition to the textual result shown in FIG. 3, users may be presented with an aggregated graphical view of the optimal maintenance schedule of a group of assets. As an additional example, Prognostics was applied to the engine oil of six Caterpillar 785D dump trucks, and a representation of how the optimal maintenance schedules may be displayed is shown in FIG. 4. Here, each bar represents the asset numbered on the y-axis, and the time of continued operation is shown on the x-axis. Each timeline shows the predicted optimal maintenance requirements with continued operation on the oil currently in each asset, where zero on the x-axis represents the most recent measurement of that asset. For example, if asset 347 (top bar) was just measured, it would require no maintenance action for 280 hours of continued operation. After 300 hours it is expected that the oil will require changing, and after 650 hours, there is a 50% chance that the oil will have degraded severely. Between 280 and 300 hours the software recommends that new samples should be collected after which the model and recommendations will be updated based on the new information. Operating according to the prescribed timeline will allow the user to avoid exceeding their risk tolerance for severely degraded oil. Asset 326 (3rd from top) has already exceeded the user-defined risk so an immediate oil change is recommended.

When data from multiple assets are sent to Prognostics, they can be analyzed collectively as a fleet, and this is how the results in FIG. 4 were obtained. Typically, fleet analysis will require fewer samples per asset than individual asset analysis because data accumulate faster. Costs associated with corrective and preventative oil change and cost of sampling can be provided in place of risk tolerance, and can be used to determine a maintenance schedule that optimizes overall cost.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

1. An on-site oil analysis method comprising:
using a rule-based diagnostic system to generate historical oil condition indicators for an asset;
using said historical oil condition indicators to model future oil condition indicators including probabilistic trajectories of continued oil degradation;
determining, based on a user risk tolerance level, when a modeled future oil condition indicator will result in oil failure;
detecting the oil condition indicator which will result in oil failure first and when; and
outputting at least the determined oil condition indicator which will result in oil failure first and when.

2. The method of claim 1 in which modeling future oil condition indicators include employing a stochastic degradation model.

3. The method of claim 1 in which the oil condition indicators include a Si concentration, an Fe concentration, a Cu concentration, a Na concentration, a Pb concentration, an Al concentration, a Cr concentration, viscosity, particle count, water concentration, oxidation, TAN, and/or TBN.

4. The method of claim 1 in which the rule based diagnostic system also generates historical oil change data.

5. The method of claim 1 further including determining and outputting when a new measurement is required for an oil condition indicator and when an oil change is recommended.

6. The method of claim 1 further including determining for a fleet of assets wherein oil change is required.

7. An on-site oil analysis system comprising:
a rule-based diagnostic system for generating historical oil condition indicators for an asset;
means for modeling future oil condition indicators including probabilistic trajectories of continued oil degradation using said historical oil condition indicators;
means for determining, based on a user risk tolerance level, when a 6 modeled future oil condition indicator will result in oil failure;
means for detecting the oil condition indicator which will result in oil failure first and when; and
means for outputting at least the determined oil condition indicator which will result in oil failure first and when.

8. The system of claim 7 in which the means for modeling future oil condition indicators include employing a stochastic degradation model.

9. The system of claim 7 in which the oil condition indicators include a Si concentration, an Fe concentration, a Cu concentration, a Na concentration, a Pb concentration, an Al concentration, a Cr concentration, viscosity, particle count, water concentration, oxidation, TAN, and/or TBN.

10. The system of claim 7 in which the rule based diagnostic system is also configured to generate historical oil change data.

11. The system of claim 7 further including means for determining and outputting when a new measurement is required for an oil condition indicator and when an oil change is recommended.
